(19) United States
(12) Patent Application Publication
(10) Pub. No.: US 2016/0158202 A1
(43) Pub. Date: Jun. 9, 2016

(54) Low Dose Pharmaceutical Composition

(71) Applicant: Cipla Limited, Mumbai (IN)

(72) Inventors: Geena MALHOTRA, Mumbai (IN); Shrinivas Madhukar PURANDARE, Mumbai (IN)

(21) Appl. No.: 15/047,091

(22) Filed: Feb. 18, 2016

Related U.S. Application Data
(63) Continuation of application No. 14/890,235, filed on Nov. 10, 2015.
Application No. 14/890,235 is a continuation-in-part of application No. 13/825,471, filed on Apr. 19, 2013.

(30) Foreign Application Priority Data
Oct. 1, 2010 (IN) 2750/MUM/2010
May 10, 2013 (IN) 1696/MUM/2013

(51) Int. Cl. A61K 31/4196; A61K 9/20; A61K 9/28; A61K 9/00; A61K 9/14

(52) U.S. Cl. CPC A61K 31/4196; A61K 9/0053; A61K 9/14; A61K 9/28; A61K 9/20

(57) ABSTRACT

This invention provides a low dose pharmaceutical composition comprising deferasirox or a pharmaceutically acceptable derivative thereof and one or more pharmaceutically acceptable excipients. A unit dose of the pharmaceutical composition comprises from about 50 mg to about 100 mg of deferasirox, from about 150 mg to about 200 mg of deferasirox or from about 260 mg to about 350 mg of deferasirox. The pharmaceutical composition of the present invention, wherein the pharmaceutical composition comprises deferasirox, may be used to treat chronic iron overload or to treat lead toxicity. The pharmaceutical composition of the present invention, wherein the pharmaceutical composition comprises deferasirox and deferiprone, may be used to treat lead toxicity. This invention also provides a process for preparing the low dose pharmaceutical composition, the process comprising: dissolving or adsorbing or blending deferasirox and at least one excipient to produce a dispersion of deferasirox; and processing the dispersion to produce a desired dosage form.

### CROSS-REFERENCE TO RELATED APPLICATIONS

This application is a continuation of and claims priority to U.S. patent application Ser. No. 14/890,235 filed Nov. 10, 2015, and entitled “Low Dose Pharmaceutical Composition,” which is a filing under 35 U.S.C. 371 of International Application No. PCT/GB2014/051400 filed May 8, 2014, entitled “Low Dose Pharmaceutical Composition,” which claims priority to Indian Patent Application No. 1696/MUM/2013 filed May 10, 2013. This application is also a continuation in part of and claims priority to U.S. patent application Ser. No. 13/825,471 filed Apr. 19, 2013, published as U.S. Patent Application Publication No. US 2014/0147503 A1, and entitled “Pharmaceutical Composition Comprising Deferasirox,” which is a filing under 35 U.S.C. 371 of International Application No. PCT/GB2011/001428 filed Sep. 30, 2011, entitled “Pharmaceutical Composition,” which claims priority to Indian Patent Application No. 2750/MUM/2010 filed Oct. 1, 2010. These applications are incorporated by reference herein in their entirety.

### FIELD OF INVENTION

The present invention relates to a low dose pharmaceutical composition comprising an iron chelating agent. The present invention also provides a process of preparing such low dose pharmaceutical composition and its use in the treatment of chronic iron overload.

### BACKGROUND OF INVENTION

Deferasirox has the chemical name 4-[3,5-bis(2-hydroxyphenyl)-[1, 2, 4]triazol-1-yl]enzoic acid and is reported to have the following chemical structure.

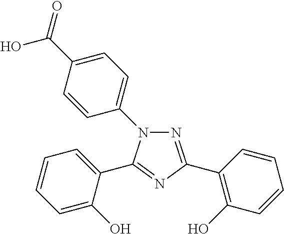 [SMILES: O=C(O)c1ccc(-n2nc(-c3ccccc3O)nc2-c2ccccc2O)cc1]

Deferasirox is an orally active iron chelator and has been approved for the treatment of iron overload in transfusion dependent anemias (transfusion hemosiderosis) in particular thalassemia major, thalassemia intermediate and in sickle cell disease to reduce iron-related morbidity and mortality in patients having an age of two years and older.

Chronic iron overload is a result of regular blood transfusions used in the treatment of several conditions including β-thalassemia, sickle cell disease and myelodysplastic syndromes.

Each unit of blood contains iron and as the human body has no physiological mechanism to actively excrete excess iron, repeated blood transfusions result in excessive accumulation of iron. This excess of iron deposited in body tissues can cause severe damage to organs such as liver, heart, endocrine organs. This may lead to many complications including cardiomyopathy, liver cirrhosis, diabetes mellitus and reduced life expectancy.

Deferasirox mobilizes tissue iron by forming soluble stable complexes that are then excreted in the feces. It is a tridentate iron chelator requiring two molecules of the drug to form a stable complex. Iron is chelated both from the reticuloendothelial cells (RE cells) as well as various parenchymal tissues. The chelated iron is cleared by the liver and excreted through the bile. It also has the ability to prevent the myocardial cell iron uptake by removing iron directly from myocardial cells.

Deferasirox is highly water insoluble and is highly lipid-soluble and is also observed to possess good permeability. According to the Bio-pharmaceutics Classification System (BCS), it has been classified as a Class II drug, implying that it is a poorly soluble, and a highly permeable drug. Though deferasirox is highly water insoluble, whatever limited solubility it has, that too exhibits a high pH-dependent solubility. Though it is practically insoluble in lower pH, even at a pH of 6.8, it still remains insoluble, until the buffer strength is altered to get optimal dissolution profile.

Deferasirox being practically insoluble in aqueous media generally exhibits a poor dissolution profile and consequently poor bioavailability.

Several strategies and formulations have been employed to overcome these limitations of solubility and poor bioavailability. Although existing strategies such as complexing drugs with cyclodextrins, conjugation to dendrimers, salt formation of ionizable drugs and the use of co-solvents have been shown to improve drug solubility, solubilization methods that can improve the absorption of the drug are still highly desirable.

Deferasirox is commercially available as dispersible tablet (EXJADE®) for oral administration. EXJADE is supplied as a dispersible tablet containing 125 mg, 250 mg and 500 mg of deferasirox per tablet. This tablet is dispersed in a glass of water or any other suitable drink, and this resulting suspension is then administered to the patient.

Deferasirox is administered as a once daily oral iron chelator, which is prescribed as a dispersible tablet, i.e., a tablet which needs to be dispersed in an aqueous medium prior to administration.

Deferasirox is typically administered at an initial dose of about 20 mg/kg body weight, and the dose is adjusted up to a maximum of 40 mg/kg body weight, which means that, the recommended dosage of deferasirox is on the higher side in order to have a clinical benefit.

More specifically, in transfusional overload, the initial dose of deferasirox is 20 mg/kg, not exceeding 40 mg/kg once daily, which ultimately amounts to an intake of 3-6 tablets of EXJADE®.

In non-transfusion-dependent thalassemia (NTDT), the initial dose of deferasirox is 10 mg/kg, not exceeding 20 mg/kg once daily, which ultimately amounts to an intake of 2-3 tablets of EXJADE®.

Premarketing studies have demonstrated elevations in liver transaminases in almost ⅓ of patients. While these initial reports documented only non-sustained elevations, in September 2007, the FDA updated post market safety findings of this agent, previously documenting incidents of renal failure, to include adverse hepatic events, including drug-induced hepatitis and liver failure.

There have been a few post marketing notifications of hepatic failure some with a fatal outcome to the FDA. Most of these events have occurred in patients greater than 55 years of age with significant co-morbidities including liver cirrhosis and multiorgan failure.

Mitochondrial injury is one of the possible mechanisms of deferasirox-induced liver injury. Hallmark of this type of injury is microvesicular fat in hepatocytes that can revolve into macrovesicular lesions, focal necrosis, fibrosis, and cholestasis consistent with this patient's liver biopsy. Furthermore, patients often experience nonspecific symptoms of insidious onset, such as nausea, vomiting, fatigue, and weight loss, while jaundice is a late finding. Hence, extreme caution should be taken in using deferasirox in patients who have underlying liver disease.

Renal toxicity is a relatively frequent adverse event in patients receiving deferasirox treatment, with proximal tubular dysfunction and a decreased Glomerular Filtration Rate. Clinicians have to regularly assess their patients to prevent chronic renal injury that may result from a prolonged tubular injury. Long-term follow-up is therefore needed.

Further, Fanconi Syndrome is associated with the use of deferasirox. Fanconi Syndrome is a generalized disturbance of proximal tubular function leading to renal losses of glucose, phosphate, calcium, uric acid, amino acids, bicarbonates, and other organic compounds.

Acute interstitial nephritis was also observed in a patient treated with deferasirox for myelodysplastic syndrome.

As side effects are not uncommon with the use of deferasirox, optimal therapy is always required to achieve the best clinical outcomes while minimizing these side effects.

Further, deferasirox is recommended to be taken daily on an empty stomach at least 30 minutes before food, preferably at the same time each day.

That means the pharmacokinetic properties of deferasirox are affected by the prandial status of a patient receiving the treatment, i.e. it exhibits a “food effect”.

Accordingly, the patients receive specific instructions to administer deferasirox on an empty stomach. Hence, deferasirox is administered in a fasting state in an attempt to minimize the food effect. Administration of a deferasirox composition with food may change its bioavailability by affecting either the drug substance or the composition in which the drug substance is formulated.

This situation is unsatisfactory and inconvenient to the thalassemia patients undergoing treatment with deferasirox since their medication usually consists of multiple tablets.

The general therapy regimen and its administration limitation such as food effect are unavoidable. Also, to achieve the maximal effect of the administered medications it would be necessary to consider the bioavailability of the administered drugs to achieve the desired effect failing which such therapies and drug regimens would be futile and would also be taxing to the morbid state of the patients.

Accordingly, there have been no prior art disclosures of compositions of deferasirox that are free of the food effect and which thereby facilitate patient compliance and superior bioavailability. The currently commercialized dosage form and the recommended dose still do not address the unsolved tribulations of the deferasirox therapy.

WO 2004035026 discloses a dispersible tablet of deferasirox wherein the active ingredient is present in an amount of from 5% to 40% by weight based on total weight of the tablet.

WO 2005097062 discloses a dispersible tablet of deferasirox wherein the active ingredient is present in an amount of from 42% to 65% by weight based on total weight of the tablet.

WO 2007045445 discloses a dispersible tablet of deferasirox or a pharmaceutically acceptable salt thereof present in an amount of from 42% to 65% by weight based on total weight of the tablet and at least one pharmaceutically acceptable excipient suitable for the preparation of dispersible tablets and to process for making said dispersible tablet.

WO 2009067557 discloses a process of preparing deferasirox formulations having sufficiently high dissolution rate and good bioavailability wherein said process comprises co-milling deferasirox with at least two pharmaceutically acceptable excipients in the absence of any solvent.

WO 2010035282 discloses oral pharmaceutical composition comprising deferasirox in the form of a dispersible tablet wherein the active ingredient has a mean particle size less than about 100 μm and is present in an amount greater than 66% by weight based on total weight of the tablet.

WO 2012/042224 discloses a pharmaceutical composition comprising deferasirox in the form of particles wherein the particles have an average particle size of less than or equal to about 2000 nm.

Deferasirox Induced Liver Injury in Haemochromatosis, Journal of the College of Physicians and Surgeons Pakistan 2010, Vol. 20 (8): 551-553, Naeem Aslam, Parveen Mettu, Luis S. Marsano-Obando and Anthony Martin explains that drug-induced liver injury is a common side-effect of many medicines and is particularly problematic when the original condition under treatment is already causing liver damage. In particular, this article describes the hepatotoxicity induced by deferasirox in a patient with haemochromatosis with a discussion of possible pathogenetic mechanism.

Acute interstitial nephritis due to deferasirox: a case report, Nephrol. Dial. Transplant (2008) 23 (10): 3356-3358, Godela Brosnahan, Neriman Gokden and Sundararaman Swaminathan describes the case of a 62 year-old man with myelodysplastic syndrome who developed a progressive decline in renal function after starting deferasirox. A kidney biopsy showed acute interstitial nephritis with increased eosinophils, suggesting drug hypersensitivity. Deferasirox was discontinued and renal function returned to baseline.

Deferasirox-induced renal impairment in children: an increasing concern for pediatricians. Pediatric Nephrology, 2012 November; 27(11):2115-22, Dubourg L, Laurain C, Ranchin B, Pondarré C, Hadj-Aïssa A, Sigaudo-Roussel D, Cochat P evaluated tubular and glomerular function before and after the initiation of deferasirox therapy in a pediatric patient population and found that renal toxicity was a frequent adverse event. The article advised that routine renal assessment was required to prevent chronic kidney disease that could result from prolonged tubular injury.

Acute renal failure and Fanconi syndrome due to deferasirox, Nephrol. Dial. Transplant (2010) 25 (7): 2376-2378, Steven Grangé, Dominique M. Bertrand, Dominique Guerrot, Florence Eas, Michel Godin explains that recent data from large studies have confirmed the renal toxicity of deferasirox. This article reports a case of Fanconi syndrome associated with acute renal failure in a patient receiving deferasirox.

Combined chelation of lead (II) by deferasirox and deferiprone in rats as biological model, Biometals (2014), 27:89-95, F. Dahooee Balooch et al investigated the capability of deferasirox and deferiprone in removing lead from the body. Rats were dosed with lead for 45 days then received chelation therapy with deferasirox and deferiprone for 10 days to reduce the lead levels. Combined chelation therapy showed higher efficacy and lower toxicity than single therapies.

Various formulations that are disclosed and that are available in the market contain a dose of 20 mg/kg body weight and a maximum of 40 mg/kg body weight.

Although deferasirox is the drug of choice for the treatment of thalassemia, administration of deferasirox for a longer duration and in higher doses to achieve the desired clinical effects may result in serious side effects. Accordingly, patients need to be regularly monitored for vital organs such as heart, endocrine organs (thyroid, testes, ovaries, and pancreas) and the liver but additional attention needs to be given to regular monitoring of renal function in patients who are at an increased risk of complications or on chelator therapy.

The possible strategies for optimizing deferasirox therapy and ultimately reducing the side effects may include applying alternate day treatment or allowing a washout period or using deferasirox in combination with other iron chelators. However, large and detailed clinical studies would be required to verify these strategies.

Considering the existing variety, deferasirox compositions with low dose could be the best available option. However, no composition is yet available which includes low dose deferasirox, wherein the total daily dose of deferasirox is less than the conventionally administered daily dose, and which is equally effective for the treatment of chronic iron overload.

Hence, to achieve a promising result with the administration of deferasirox for the desired indications and with minimal side effects, there is a need to develop low dose compositions wherein the total daily dose of deferasirox is less than the conventionally administered daily dose and which is equally effective for the treatment of chronic iron overload.

Further, to overcome the food effect, the inventors of the present invention have designed formulations comprising deferasirox which reduce or nullify the food effect to ensure better bioavailability. Such formulations of deferasirox are patient compliant, robust, and stable and also exhibit optimal dissolution properties.

The above drawbacks and rationales have lead the inventors of the present invention to develop pharmaceutical compositions comprising a reduced dose or a low dose of deferasirox further exhibiting improved bioavailability, and exhibiting reduced or no food effect, without causing dose related side effects and which also can be prepared in an easy and cost-effective manner. These pharmaceutical compositions comprising a low dose of deferasirox further exhibit equally acceptable dissolution properties and absorption properties thus leading to better bioavailability.

### OBJECT OF THE INVENTION

An object of the present invention is to provide a low dose pharmaceutical composition comprising deferasirox along with one or more pharmaceutically acceptable excipients.

Another object of the present invention is to provide a low dose pharmaceutical composition comprising deferasirox wherein the total daily dose of deferasirox is less than the conventionally administered daily dose.

Yet another object of the present invention is to provide a low dose pharmaceutical composition comprising deferasirox exhibiting reduced side effects.

Another object of the present invention is to provide a low dose pharmaceutical composition comprising deferasirox exhibiting improved bioavailability.

Another object of the present invention is to provide a low dose pharmaceutical composition comprising deferasirox exhibiting minimal or no food effect.

Yet another object of the present invention is to provide a process for preparing the low dose pharmaceutical composition of deferasirox.

Yet another object of the present invention is to provide a low dose pharmaceutical composition comprising deferasirox for use in the treatment of chronic iron overload.

Yet another object of the present invention is to provide a low dose pharmaceutical composition comprising deferasirox for use in the treatment of lead toxicity.

Another object of the present invention is to provide a low dose pharmaceutical composition comprising deferasirox and deferiprone for use in the treatment of lead toxicity.

A further object of the present invention is to provide a method for the treatment of chronic iron overload which comprises administering a low dose pharmaceutical composition comprising deferasirox.

Yet another object of the present invention is to provide a method for the treatment of lead toxicity which comprises administering a low dose pharmaceutical composition comprising deferasirox.

Another object of the present invention is to provide a method for the treatment of lead toxicity which comprises administering a low dose pharmaceutical composition comprising deferasirox and deferiprone.

### SUMMARY OF THE INVENTION

According to one aspect of the present invention, there is provided a low dose pharmaceutical composition comprising deferasirox.

According to one aspect of the present invention, there is provided a low dose pharmaceutical composition comprising deferasirox or a pharmaceutically acceptable derivative thereof and one or more pharmaceutically acceptable excipients.

According to one aspect of the present invention, there is provided a low dose pharmaceutical composition comprising deferasirox exhibiting reduced side effects.

According to one aspect of the present invention, there is provided a low dose pharmaceutical composition comprising deferasirox exhibiting improved bioavailability.

According to another aspect of the present invention, there is provided a low dose pharmaceutical composition comprising deferasirox exhibiting minimal or no food effect.

According to another aspect of the present invention there is provided a process for preparing the low dose pharmaceutical composition comprising deferasirox.

According to one aspect of the present invention there is provided a process for preparing the low dose pharmaceutical composition comprising

dissolving or adsorbing or blending deferasirox and at least one excipient to produce a dispersion of deferasirox; and

processing the dispersion to produce a desired dosage form.

According to yet another aspect of the present invention there is provided a low dose pharmaceutical composition comprising deferasirox for use in the treatment of chronic iron overload.

According to yet another aspect of the present invention there is provided a low dose pharmaceutical composition comprising deferasirox for use in the treatment of lead toxicity.

According to another aspect of the present invention there is provided a low dose pharmaceutical composition comprising deferasirox and deferiprone for use in the treatment of lead toxicity.

According to a further aspect of the present invention there is provided a method for the treatment of chronic iron overload which comprises administering a low dose pharmaceutical composition comprising deferasirox.

According to a further aspect of the present invention there is provided a method for the treatment of lead toxicity which comprises administering a low dose pharmaceutical composition comprising deferasirox.

According to another aspect of the present invention there is provided a method for the treatment of lead toxicity which comprises administering a low dose pharmaceutical composition comprising deferasirox and deferiprone.

The present invention provides a pharmaceutical composition comprising deferasirox or a pharmaceutically acceptable derivative thereof and one or more pharmaceutically acceptable excipients. Said pharmaceutical composition may comprise any of the features described below, including the quantity of deferasirox in a unit dose, the excipients present in the composition, the particle size of the deferasirox, its use in the treatment of chronic iron overload and its use in providing a specific daily dose of deferasirox.

### BRIEF DESCRIPTION OF THE DRAWINGS

FIG. 1—Mean Plasma Concentrations Vs Time Curve for Test Product (T) and Reference Product (R) of Deferasirox on linear scale.

FIG. 2—Mean Plasma Concentrations Vs Time Curve for Test Products (T1 or Test A and T2 or Test B) and Reference Product (R) of Deferasirox on linear scale.

FIG. 3—Mean Plasma Concentrations Vs Time Curve for Test Products (T1, T2 and T3) and Reference Product (R) of Deferasirox on linear scale.

FIG. 4—Mean Plasma Concentrations Vs Time Curve for Test Product (T) and Reference Product (R) of Deferasirox iron complex on linear scale.

FIG. 5—Mean Plasma Concentrations Vs Time Curve for Test Product (T) and Reference Product (R) of Deferasirox on linear scale.

### DETAILED DESCRIPTION OF THE INVENTION

Deferasirox has been conventionally administered at a dose 20 mg/kg body weight and a maximum of 40 mg/kg body weight for the treatment of chronic iron overload.

The death rate and indiscriminate use of deferasirox is massive affecting the safety of all patients since no sufficient or effective safeguards seem to have been implemented so far to reduce toxicity of deferasirox.

Further, deferasirox is noted to exhibit “food effect”. That means the bioavailability of deferasirox depends on whether it was administered in a fed or fasted condition.

Thus there is a dire need to develop compositions which address the food effect issues of deferasirox.

The inventors of this invention have made an effort to formulate a low dose pharmaceutical compositions of deferasirox, which can also be effectively administered for the treatment of chronic iron overload. Furthermore, the low dose compositions of the present invention have improved bioavailability, exhibit reduced or no food effect, as well as are easy to formulate while being cost-effective.

The term “low dose” as used herein refers to a therapeutically effective dose of deferasirox, which is less than the conventional dose required to produce the therapeutic effect.

The term “unit dose” or “single unit dose” as used herein refers to one discrete pharmaceutical dosage form.

Suitably, a low dose formulation is one in which a unit dose comprises less deferasirox than the conventional unit dose. For example, a unit dose or single unit dose of the low dose formulation may comprise from about 50 mg to about 100 mg of deferasirox, from about 150 mg to about 200 mg of deferasirox or from about 260 mg to about 350 mg of deferasirox. Such a unit dose or single unit dose conveniently enables a patient to be provided with less than the conventional total daily dose of deferasirox. For example, such a unit dose or single unit dose may enable a patient to be provided with from about 0.1 mg/kg body weight to less than about 20 mg/kg body weight, which is less than the conventionally administered dose of EXJADE®.

More specifically, in transfusional overload, the low dose of deferasirox according to the present invention ranges from about 5 mg/kg to less than about 30 mg/kg.

In non-transfusion-dependent thalassemia (NTDT), the low dose of deferasirox according to the present invention ranges from about 3 mg/kg to about 15 mg/kg.

The term “Deferasirox” is used in broad sense to include not only “Deferasirox” per se but also its pharmaceutically acceptable derivatives thereof. Suitable derivatives include pharmaceutically acceptable salts, pharmaceutically acceptable solvates, pharmaceutically acceptable hydrates, pharmaceutically acceptable anhydrates, pharmaceutically acceptable enantiomers, pharmaceutically acceptable esters, pharmaceutically acceptable isomers, pharmaceutically acceptable polymorphs, pharmaceutically acceptable prodrugs, pharmaceutically acceptable tautomers, pharmaceutically acceptable complexes etc.

The term “particle size” as used herein refers to the average particle size of deferasirox. The average particle size of deferasirox may be greater than or equal to about 0.001 μm or 1 nm but less than or equal to about 10 μm or 10,000 nm.

Preferably, the average particle size of deferasirox is greater than about 1 μm or 1,000 nm but less than or equal to about 30 μm or 30,000 nm, optionally greater than about 1 μm or 1,000 nm but less than or equal to about 8 μm or 8,000 nm, greater than about 2 μm or 2,000 nm but less than or equal to about 30 μm or 30,000 nm, greater than about 2 μm or 2,000 nm but less than or equal to about 8 μm or 8,000 nm, greater than or equal to about 2.5 μm or 2,500 nm but less than or equal to about 7 μm or 7,000 nm, greater than or equal to about 2.5 μm or 2,500 nm but less than or equal to about 5 μm or 5,000 nm, or greater than or equal to about 3 μm or 3,000 nm but less than or equal to about 6 μm or 6,000 nm.

Optimization of the particle size of deferasirox can help provide a lower maximum concentration (Cₘₐₓ) of deferasirox thereby reducing side effects, reducing or nullifying the food effect and can help increase bioavailability of deferasirox thereby enabling a reduction in daily dose.

According to one aspect of the present invention, there is provided a low dose pharmaceutical composition comprising deferasirox with one or more pharmaceutically acceptable excipients wherein the total daily dose of the deferasirox is from about 0.1 mg/kg body weight to less than about 20 mg/kg body weight.

Preferably, total daily dose of the deferasirox is from about 1 mg/kg to less than about 30 mg/kg of body weight, optionally from about 1 mg/kg to less than about 20 mg/kg of body weight or from about 1 mg/kg to about 15 mg/kg of body weight or from about 1 mg/kg to about 10 mg/kg of body weight or from about 1 mg/kg to about 5 mg/kg of body weight or from about 2 mg/kg to less than about 30 mg/kg of body weight or from about 2 mg/kg to less than about 20 mg/kg of body weight or from about 2 mg/kg to about 15 mg/kg of body weight or from about 2 mg/kg to about 10 mg/kg of body weight or from about 2 mg/kg to about 5 mg/kg of body weight or from about 3 mg/kg to less than about 30 mg/kg of body weight or from about 3 mg/kg to less than about 20 mg/kg of body weight or from about 3 mg/kg to about 15 mg/kg of body weight or from about 3 mg/kg to about 10 mg/kg of body weight or from about 3 mg/kg to about 5 mg/kg of body weight or from about 5 mg/kg to less than about 30 mg/kg of body weight or from about 5 mg/kg to less than about 20 mg/kg of body weight or from about 5 mg/kg to about 15 mg/kg of body weight or from about 5 mg/kg to about 10 mg/kg of body weight.

The low dose pharmaceutical composition comprising deferasirox, according to the present invention may be administered at least once a day, optionally once a day, twice a day or three times a day.

According to another aspect of the present invention, there is provided a low dose pharmaceutical composition comprising deferasirox, wherein the unit dose or single unit dose of the pharmaceutical composition comprises from about 50 mg to about 100 mg of deferasirox, from about 150 mg to about 200 mg of deferasirox or from about 260 mg to about 350 mg of deferasirox.

Preferably, the unit dose or single unit dose of the pharmaceutical composition comprises 75 mg, 150 mg or 300 mg of deferasirox.

The low dose pharmaceutical composition, according to the present invention, may exhibit bioavailability to an extent to produce the desired pharmacological effects along with reduced side effects after dosing in a subject.

The low dose pharmaceutical composition, according to the present invention, may be used for the treatment of chronic iron overload.

The term “pharmaceutical composition” includes low dose pharmaceutical compositions of deferasirox for oral administration, such as solid dosage forms, but not limited to tablets (single layer, bilayer, multilayer, tablet in tablet and the like) which may be uncoated, film coated, sugar coated, powder coated, enteric coated, seal coated, capsules (filled with powders, powders for reconstitution, pellets, beads, mini-tablets, pills, micro-pellets, small tablet units, film coated tablets, MUPS, film coated tablets MUPS, orally disintegrating MUPS, disintegrating tablets, dispersible tablets, granules, microspheres, nanoparticles, and the like or combinations thereof), soft gelatin capsules, sachets (filled with powders, pellets, beads, mini-tablets, pills, micro-pellets, small tablet units, film coated tablets, MUPS, film coated tablets MUPS, orally disintegrating MUPS, disintegrating tablets, dispersible tablets, granules, microspheres, nanoparticles, and the like or combinations thereof) and sprinkles however other dosage forms such as liquid dosage forms (liquids, liquid dispersions, suspensions, solutions, emulsions, micro emulsions, sprays, spot-on and the like), solid dispersion, injection preparations, gels, aerosols, ointments, creams, controlled release formulations, lyophilized formulations, modified release formulations, delayed release formulations, extended release formulations, pulsatile release formulations, dual release formulations and the like may also be envisaged under the ambit of the invention.

Preferably, the pharmaceutical composition, according to the present invention is in the form of a solid oral dosage form. More preferably, the pharmaceutical composition, according to the present invention is in the form of a tablet. Most preferably, the pharmaceutical composition, according to the present invention is in the form of a dispersible tablet.

The low dose pharmaceutical compositions according to the present invention may comprise carriers/excipients suitable for formulating the same.

Accordingly, the low dose pharmaceutical composition according to the present invention may comprise one or more excipients such as, a surfcatant, solubilizer, an anticaking agent, a buffer, a polymer, a sweetener, solvents, co-solvents, a vehicle, a viscosity enhancing agent, a carrier, an adsorbent, a channeling agent, an opacifier, a diluent, a filler, a glidant, an anti-adherent, a binder, a disintegrant and a lubricant.

Suitable amphoteric, non-ionic, cationic or anionic surfactants or wetting agents may also be used in the low dose pharmaceutical compositions of the present invention.

According to the present invention, the surfactants may comprise one or more of, but not limited to polysorbates such as polysorbate 20, polysorbate 40, polysorbate 60, polysorbate 80, polysorbate 65, polysorbate 85, sorbitan fatty acid esters such as Span 20, Span 40, Span 60, Span 80, Span 120; Phosal® 50 PG (Phosphatidylcholine concentrate with at least 50% PC and propylene glycol) as well as other grades of Phosal that may be envisaged under the ambit of the invention, sodium lauryl sulfate; polyethoxylated castor oil; polyethoxylated hydrogenated castor oil, sodium dodecyl sulfate (sodium lauryl sulfate), lauryl dimethyl amine oxide, docusate sodium, cetyl trimethyl ammonium bromide (CTAB), polyethoxylated alcohols, polyoxyethylene sorbitan, octoxynol, N,N-dimethyldodecylamine-N-oxide, Hexadecyltrimethyl-ammonium bromide, polyoxyl 10 lauryl ether, Brij, bile salts (sodium deoxycholate, sodium cholate), polyoxyl castor oil, Maisine, Polyoxyl 40 hydrogenated castor oil, Polyoxyl 35 castor oil, nonylphenol ethoxylate, cyclodextrins, lecithin, methylbenzethonium chloride, carboxylates, sulphonates, petroleum sulphonates, alkylbenzenesulphonates, naphthalenesulphonates, olefin sulphonates, alkyl sulphates, sulphates, sulphated natural oils and fats, sulphated esters, sulphated alkanolamides, alkylphenols, ethoxylated and sulphated, ethoxylated aliphatic alcohol, polyoxyethylene surfactants, carboxylic esters, polyethylene glycol esters, anhydrosorbitol ester and its ethoxylated derivatives, glycol esters of fatty acids, carboxylic amides, monoalkanolamine condensates, polyoxyethylene fatty acid amides, quaternary ammonium salts, amines with amide linkages, polyoxyethylene alkyl and alicyclic amines, N,N,N,N tetrakis substituted ethylenediamines, 2-alkyl-1-hydroxyethyl-2-imidazolines, N-coco 3-aminopropionic acid/sodium salt, N-tallow 3-imino-dipropionate disodium salt, N-carboxymethyl n-dimethyl n-9-octadecenyl ammonium hydroxide and n-cocoamidethyl n-hydroxyethylglycine sodium salt and the like and combinations thereof.

The amount of surfactant that may be present in the low dose pharmaceutical composition can range from about 2% to about 10%.

Suitable solubilizers according to the present invention, comprise, but are not limited to, Phosal® 50 PG (Phosphatidylcholine concentrate with at least 50% PC and propylene glycol) as well as other grades of Phosal that may be envisaged under the ambit of the invention, Maisine, Polyoxyl 40 hydrogenated castor oil, Polyoxyl 35 castor oil and the like and combinations thereof.

The amount of solubilizer that may be present in the low dose pharmaceutical composition can range from about 2% to about 15%.

Suitable anticaking agents may also be used in the present invention such as, but not limited to, hydrogenated castor oil, silica with dimethyldichlorosilane and the like and combinations thereof.

The amount of anticaking agent that may be present in the low dose pharmaceutical composition can range from about 1% to about 10%.

The buffer or the pH adjusting agent may comprise one or more of organic or inorganic acids such as, but not limited to, citric acid, citric acid monohydrate, sodium citrate, sodium citrate dihydrate, sodium hydrogen sulphate borate buffer, phosphates (sodium hydrogen orthophosphate, disodium hydrogen phosphate, Sodium dihydrogen phosphate), trometamol, acetate buffer, citrate buffer and their hydrates, equivalent conventional buffers and the like and combinations thereof.

The amount of buffer or the pH adjusting agent that may be present in the low dose pharmaceutical composition can range from about 2% to about 8%.

Suitable polymers or polymers blends, according to the present invention, may comprise one or more water soluble, water insoluble or water swellable polymers, but not limited to Water soluble polymers which may be used in the pharmaceutical antiretroviral composition of the present invention, include, but are not limited to, homopolymers and co-polymers of N-vinyl lactams, especially homopolymers and co-polymers of N-vinyl pyrrolidone e.g. polyvinylpyrrolidone (PVP), co-polymers of PVP and vinyl acetate, co-polymers of N-vinyl pyrrolidone and vinyl acetate (Copovidone) or vinyl propionate, dextrins such as grades of maltodextrin, cellulose esters and cellulose ethers, high molecular polyalkylene oxides such as polyethylene oxide and polypropylene oxide and co-polymers of ethylene oxide, propylene oxide, acrylic copolymers e.g. Eudragit E100 or Eudragit EPO; Eudragit L30D-55, Eudragit FS30D, Eudragit RL30D, Eudragit RS30D, Eudragit NE30D, Acryl-Eze, polyvinylacetate, for example, Kollicoat SR 30D, cellulose derivatives such as ethylcellulose, cellulose acetate e.g. Surelease, Aquacoat ECD and Aquacoat CPD, polyethylene oxide; poly (hydroxy alkyl methacrylate); poly (vinyl) alcohol, having a low acetal residue, which is cross-linked with glyoxal, formaldehyde or glutaraldehyde and having a degree of polymerization of from 200 to 30,000; a mixture of methyl cellulose, cross-linked agar and carboxymethyl cellulose; Carbopol® carbomer which is an acidic carboxy polymer; Cyanamer® polyacrylamides; cross-linked water swellable indene-maleic anhydride polymers; Goodrich® polyacrylic acid; starch graft copolymers; Aqua Keeps® acrylate polymer polysaccharides composed of condensed glucose units such as diester cross-linked polyglucan, and the like; Amberlite® ion exchange resins; Explotab® sodium starch glycolate; Ac-Di-Sol® croscarmellose sodium and the like or combinations thereof.

The amount of polymer that may be present in the low dose pharmaceutical composition can range from about 4% to about 30%.

Suitable sweeteners which may be used in the low dose pharmaceutical composition of the present invention, include, but are not limited to, saccharin, sodium saccharin, aspartame, acesulfame, cyclamate, alitame, a dihydrochalcone sweetener, monellin, neohesperidin, neotame, stevioside and sucralose, the pharmaceutically acceptable salts and the like and combinations thereof.

The amount of sweetener that may be present in the low dose pharmaceutical composition can range from about 2% to about 7%.

Suitable solvents/co-solvents/vehicle that may be used in the low dose pharmaceutical composition of the present invention, include, but are not limited to polysorbates such as polysorbate 20, polysorbate 40, polysorbate 60, polysorbate 80, polysorbate 65, polysorbate 85, Polyoxyl 35 castor oil, Phosal® 50 PG (Phosphatidylcholine concentrate with at least 50% PC and propylene glycol) as well as other grades of Phosal that may be envisaged under the ambit of the invention, hydrogenated castor oil, medium and/or long chain fatty acids or glycerides, monoglycerides, diglycerides, triglycerides, structured triglycerides, soyabean oil, peanut oil, corn oil, corn oil mono glycerides, corn oil di glycerides, corn oil triglycerides, polyethylene glycol, sorbitol, caprylocaproyl macroglycerides, caproyl 90, propylene glycol, polyoxyethylene sorbitan fatty acid esters, polyoxyethylene castor oil derivatives, castor oil, hydrogenated castor oil, cottonseed oil, olive oil, safflower oil, peppermint oil, coconut oil, palm seed oil, water, beeswax, oleic acid, methanol, ethanol, isopropyl alcohol, butanol, acetone, methylisobutyl ketone, methylethyl ketone, glycerol, sorbitol, glycerol monolinoleate, water and the like and combinations thereof.

The amount of solvents/co-solvents/vehicle that may be present in the low dose pharmaceutical composition can range from about 5% to about 20%.

Suitable viscosity enhancing agents that may be used in the low dose pharmaceutical composition of the present invention include, but are not limited to polymers or polymers blends as mentioned above, derivatives of sugars, such as lactose, saccharose, hydrolyzed starch (maltodextrin), hydroxypropylmethylcellulose (HPMC) and the like or combinations thereof.

Suitable carriers or adsorbents that may be used in the low dose pharmaceutical composition of the present invention include, but are not limited to various forms of silica which comprise mesoporous, nanoporous, fumed silica, carbon dioxide and the like or combinations thereof.

The amount of carriers or adsorbents that may be present in the low dose pharmaceutical composition can range from about 10% to about 70%.

Suitable channeling agents that may be used in the low dose pharmaceutical composition of the present invention include, but are not limited to sodium chloride, sugars, polyols and the like and combinations thereof.

The amount of channeling agents that may be present in the low dose pharmaceutical composition can range from about 5% to about 30%.

According to the present invention, pharmaceutically acceptable opacifier for use in the low dose pharmaceutical composition of the present invention may comprise one or more, but is not limited to titanium dioxide, xanthan gum, bentonite and the like or combinations thereof.

The amount of opacifier present in the low dose pharmaceutical composition can range from about 0.5% to about 5%.

According to the present invention, pharmaceutically acceptable diluents or fillers for use in the low dose pharmaceutical composition of the present invention may comprise one or more, but not limited to lactose, lactose monohydrate (for example, spray-dried lactose, α-lactose, β-lactose) lactose available under the trade mark Tablettose, various grades of lactose available under the trade mark Pharmatose or other commercially available forms of lactose, lactitol, saccharose, sorbitol, mannitol, dextrates, dextrins, dextrose, maltodextrin, croscarmellose sodium, silicified microcrystalline cellulose, microcrystalline cellulose (for example, microcrystalline cellulose available under the trade mark Avicel), hydroxypropylcellulose, L-hydroxypropylcellulose (low substituted), (HPMC, methylcellulose polymers (such as, for example, Methocel A, Methocel A4C, Methocel A15C, Methocel A4M), hydroxyethylcellulose, sodium carboxymethylcellulose, carboxymethylene, carboxymethyl hydroxyethylcellulose and other cellulose derivatives, starches or modified starches (including potato starch, corn starch, maize starch and rice starch) and mixtures thereof.

The amount of diluents or fillers that may be present in the low dose pharmaceutical composition can range from about 15% to about 60%.

According to the present invention, glidants, anti-adherents and lubricants may also be incorporated in the low dose pharmaceutical composition of the present invention, which may comprise one or more, but not limited to stearic acid and pharmaceutically acceptable salts or esters thereof (for example, magnesium stearate, calcium stearate, sodium stearyl fumarate or other metallic stearate), talc, waxes (for example, microcrystalline waxes) and glycerides, light mineral oil, PEG, silica acid or a derivative or salt thereof (for example, silicates, silicon dioxide, colloidal silicon dioxide and polymers thereof, crospovidone, magnesium stearate, magnesium aluminosilicate and/or magnesium alumino metasilicate), sucrose ester of fatty acids, hydrogenated vegetable oils (for example, hydrogenated castor oil), or mixtures thereof.

The amount of glidants, anti-adherents and lubricants that may be present in the low dose pharmaceutical composition can range from about 0.1% to about 5%.

According to the present invention, suitable binders may also be present in the low dose pharmaceutical composition of the present invention, which may comprise one or more, but not limited to polyvinyl pyrrolidone (also known as povidone), polyethylene glycol(s), acacia, alginic acid, agar, calcium carragenan, cellulose derivatives such as ethyl cellulose, methyl cellulose, hydroxypropyl cellulose, hydroxypropyl methyl cellulose, sodium carboxymethylcellulose, dextrin, gelatin, gum arabic, guar gum, tragacanth, sodium alginate, or mixtures thereof or any other suitable binder.

The amount of binder that may be present in the low dose pharmaceutical composition can range from about 5% to about 20%.

According to the present invention, suitable disintegrants may also be present in the low dose pharmaceutical composition of the present invention, which may comprise one or more, but not limited to hydroxylpropyl cellulose (HPC), low density HPC, carboxymethylcellulose (CMC), sodium CMC, calcium CMC, crospovidone, croscarmellose sodium; starches exemplified under examples of fillers and also carboxymethyl starch, hydroxylpropyl starch, modified starch; crystalline cellulose, sodium starch glycolate; alginic acid or a salt thereof, such as sodium alginate or their equivalents and mixtures thereof.

The amount of disintegrant that may be present in the low dose pharmaceutical composition can range from about 5% to about 40%.

The solid dosage form, according to the present invention may be coated or uncoated, but not limited to seal coating, film coating, enteric coating or a combination thereof Additional excipients such as film forming polymers, solvents, plasticizers, anti-adherents, opacifiers, colorants, pigments, antifoaming agents, and polishing agents can be used in coatings.

Suitable film-forming agents include, but are not limited to, cellulose derivatives, such as, soluble alkyl- or hydroalkyl-cellulose derivatives such as methylcelluloses, hydroxymethyl celluloses, hydroxyethyl celluloses, hydroxypropyl celluloses, hydroxymethylethyl celluloses, hydroxypropyl methylcelluloses, sodium carboxymethyl celluloses, insoluble cellulose derivatives such as ethylcelluloses and the like, dextrins, starches and starch derivatives, polymers based on carbohydrates and derivatives thereof, natural gums such as gum Arabic, xanthans, alginates, polyacrylic acids, polyvinyl alcohols, polyvinyl acetates, polyvinylpyrrolidones, polymethacrylates and derivatives thereof, chitosan and derivatives thereof, shellac and derivatives thereof, waxes, fat substances and any mixtures or combinations thereof.

Suitable enteric coating materials, include, but are not limited to, cellulosic polymers like cellulose acetate phthalates, cellulose acetate trimellitates, hydroxypropyl methylcellulose phthalates, polyvinyl acetate phthalates, methacrylic acid polymers and copolymers and any mixtures or combinations thereof.

Some of the excipients are used as adjuvant to the coating process, including excipients such as plasticizers, opacifiers, antiadhesives, polishing agents, and the like.

Suitable plasticizers include, but are not limited to, castor oil, diacetylated monoglycerides, dibutyl sebacate, diethyl phthalate, glycerin, polyethylene glycols, propylene glycols, triacetin, triethyl citrate, and mixtures thereof.

Suitable opacifiers include, but is not limited to, titanium dioxide.

Suitable anti-adhesives include, but is not limited to, talc.

Suitable polishing agents includes, but is not limited to, polyethylene glycols of various molecular weights or mixtures thereof, talc, surfactants (glycerol monostearate and poloxamers), fatty alcohols (stearyl alcohol, cetyl alcohol, lauryl alcohol and myristyl alcohol) and waxes (carnauba wax, candelilla wax and white wax) and mixtures thereof.

Suitable solvents used in the processes of preparing the pharmaceutical antiretroviral composition of the present invention, include, but are not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran, and mixtures thereof.

The particle size of deferasirox can be reduced by any process such as but not limited to milling, precipitation, homogenization, high pressure homogenization, spray-freeze drying, supercritical fluid technology, double emulsion/solvent evaporation, PRINT, thermal condensation, ultrasonication and spray drying.

The pharmaceutical compositions of the present invention comprising deferasirox can be manufactured by any of the types of processes described above. The processes as described above, however, do not limit the scope of the invention.

The deferasirox as obtained by any of the processes described above or any other processes known to a person skilled in art may further be processed to prepare the desired dosage forms.

The present invention thus provides a process for preparing a low dose pharmaceutical composition comprising deferasirox, wherein the deferasirox microemulsion, formed by dissolving deferasirox in suitable solubilizers or solvents, is processed further to obtain the desirable dosage forms such as, but not limited to, oral liquids, tablets, soft gelatin capsules or capsules of gelatin, carragenan and HPMC.

The present invention further provides a process for preparing low dose pharmaceutical compositions comprising deferasirox, wherein deferasirox is dissolved on a carrier and spray dried to obtain desirable dosage forms.

The present invention further provides a process for preparing low dose pharmaceutical compositions comprising deferasirox, wherein deferasirox is adsorbed on a carrier and spray dried to obtain desirable dosage forms.

The present invention also provides a process for preparing low dose pharmaceutical compositions comprising deferasirox obtained by the solid dispersion technique to obtain the desirable dosage forms.

The present invention also provides a process for preparing low dose pharmaceutical compositions comprising deferasirox obtained by hot melt extrusion technique to obtain the desirable dosage forms.

Further, the low dose pharmaceutical composition comprising deferasirox, according to the present invention may further comprise at least one additional active ingredient such as but not limited to desferrioxamine, deferiprone, leukotriene, probenecid, indomethacin, penicillin G, ritonavir, indinavir, saquinavir, furosemide, methotrexate, sulfinpyrazone, interferon, ribavirin, viramidine, valopicitabine, aromatase inhibitor, antiestrogen, anti-androgen, gonadorelin agonist, topoisomerase I inhibitor, topoisomerase II inhibitor, microtubule active agent, alkylating agent, anti-neoplastic, anti-metabolite, platin compound, anti-angiogenic compound, cyclooxygenase inhibitor, bisphosphonate, heparanase inhibitor, telomerase inhibitor, protease inhibitor, matrix metalloproteinase inhibitor, proteasome inhibitor, somatostatin receptor antagonist, anti-leukemic compound, ribonucleotide reductase inhibitor, S-adenosylmethionine decarboxylase inhibitor; ACE inhibitor, antibiotics such as gentamicin, amikacin, tobramycin, ciprofloxacin, levofloxacin, ceftazidime, cefepime, cefpirome, piperacillin, ticarcillin, meropenem, imipenem, polymyxin B, colistin and aztreonam; cyclosporin A, cyclosporin G, rapamycin, and combinations thereof.

The present invention further provides a low dose pharmaceutical composition comprising deferasirox for use in the treatment of chronic iron overload.

The present invention further provides a low dose pharmaceutical composition comprising deferasirox for use in the treatment of lead toxicity.

The present invention also provides a low dose pharmaceutical composition comprising deferasirox and deferiprone as an additional active ingredient for use in the treatment of lead toxicity.

The present invention further provides a method for the treatment of chronic iron overload which comprises administering a low dose pharmaceutical composition comprising deferasirox according to the present invention.

The present invention further provides a method for the treatment of lead toxicity which comprises administering a low dose pharmaceutical composition comprising deferasirox according to the present invention.

In a further embodiment, the present invention also provides a method for the treatment of lead toxicity which comprises administering a low dose pharmaceutical composition comprising deferasirox and deferiprone as an additional active ingredient.

The following examples are for the purpose of illustration of the invention only and are not intended in any way to limit the scope of the present invention.

### Example 1

### Low Dose Deferasirox Micro Emulsion—

a) Oral Liquid

| Sr. No. | Ingredients | Qty | |
|---|---|---|---|
| 1. | Deferasirox | 50-500 | mg |
| 2. | Polyoxyl 40 hydrogenated castor oil | 5 | gm |
| 3. | Phosal* | 2 | gm |
| 4. | Sodium Citrate | 50 | mg |
| 5. | Sodium Saccharin | 10 | mg |

| | | |
|---|---|---|
| 6. | Propylene Glycol/Sorbitol/Purified water | q.s. |

*Phosphatidylcholine concentrate with at least 50% PC and propylene glycol

Process:

1. Polyoxyl 40 hydrogenated castor oil and Phosal were heated.
2. Deferasirox was added to the liquid obtained in step (1).
3. Sodium citrate and sodium saccharin were dissolved in propylene glycol/sorbitol/purified water.
4. Deferasirox solution obtained in step (2) was added to the solution in step (3) to obtain the microemulsion

b) Soft Gelatin Capsules

| Sr. No. | Ingredients | Qty |
|---|---|---|
| 1. | Deferasirox | 50-125 mg |
| 2. | Polyoxyl 35 Castor Oil | 175 mg-600 mg |
| 3. | Tween 20 | 175 mg-600 mg |

Process:

1. Polyoxyl 35 Hydrogenated Castor Oil was heated.
2. Deferasirox was added to the liquid obtained in step (1).
3. The clear solution obtained in step (2) was formulated as a soft gelatin capsule.

c) Soft Gelatin Capsules

| Sr. No. | Ingredients | Qty |
|---|---|---|
| 1. | Deferasirox | 50-125 mg |
| 2. | Polyoxyl 35 Castor Oil | 350-500 mg |
| 3. | Maisine/Castor Oil/Phosal* | 400-750 mg |

*Phosphatidylcholine concentrate with at least 50% PC and propylene glycol

Process:

1. Polyoxyl 35 Hydrogenated Castor Oil was heated.
2. Deferasirox was added to the liquid obtained in step (1).
3. Maisine/Castor Oil/Phosal was added to the liquid obtained in step (2).
4. The clear solution obtained in step (3) was formulated as a soft gelatin capsule.

d) Hard Gelatin Capsules

| Sr. No. | Ingredients | Qty |
|---|---|---|
| 1. | Deferasirox | 50-125 mg |
| 2. | Polyoxyl 35 Castor Oil | 300-500 mg |
| 3. | Maisine/Castor Oil/Phosal* | 200-600 mg |
| 4. | Hydrogenated Castor Oil | 50 mg |

*Phosphatidylcholine concentrate with at least 50% PC and propylene glycol

Process:

1. Polyoxyl 35 Castor Oil and Maisine/Castor oil/Phosal were mixed.
2. Deferasirox was added to the liquid obtained in step (1).
3. Hydrogenated Castor Oil was added to liquid obtained in step (2).
4. The liquid obtained in step (3) was then blended and formulated into hard gelatin capsules.

### Example 2

### Low Dose Nanoparticulate Deferasirox Using Nanoporous Silica

a) Tablets

| Sr. No | Ingredient | Qty |
|---|---|---|
| 1. | Deferasirox | 125 mg |
| 2. | Nanoporous Silica | 320 mg |
| 3. | Methanol | q.s. |
| 4. | Silicified MCC | 95 mg |
| 5. | Sodium Chloride | 30 mg |
| 6. | Crospovidone | 37 mg |
| 7. | Magnesium Stearate | 1 mg |
| | Total | 608 mg |

Process:

1. Deferasirox was dissolved in methanol to obtain a clear solution.
2. Nanoporous silica was added to the solution obtained in step (1).
3. The solution obtained in step (2) was spray dried and the powder was then blended with pre sifted silicified MCC, sodium chloride and crospovidone
4. The blend obtained in step (3) was lubricated using pre-sifted magnesium stearate and compressed into tablets.

b) Tablets

| Sr. No | Ingredient | Qty |
|---|---|---|
| 1. | Deferasirox | 125 mg |
| 2. | Nanoporous Silica | 320 mg |
| 3. | Methanol | q.s. |
| 4. | Lactose Monohydrate | 90 mg |
| 5. | Crospovidone | 25 mg |
| 6. | Silicified MCC | 45 mg |
| 7. | Sodium Chloride | 30 mg |
| 8. | Crospovidone | 12 mg |
| 9. | Magnesium Stearate | 1 mg |
| | Total | 648 mg |

Process:

1. Deferasirox was dissolved in methanol under stirring to obtain a clear solution.
2. Nanoporous silica was added to the solution obtained in step (1) which was then sprayed onto a mixture of Lactose Monohydrate and crospovidone.
3. The deferasirox granules obtained in step (2) was then blended with silicified MCC, sodium chloride and crospovidone.
4. The blend obtained in step (3) was then lubricated using magnesium stearate and then compressed into tablets.

### Example 3

### Low Dose Nanoparticulate Deferasirox Using Supercritical Fluid Technique

a) Tablets

| Sr. No | Ingredient | Qty |
|---|---|---|
| 1. | Deferasirox | 125 mg |
| 2. | Carbon Dioxide | q.s. |
| 3. | Silicified MCC | 95 mg |
| 4. | Crospovidone | 37 mg |
| 5. | Sodium Chloride | 30 mg |
| 6. | Magnesium Stearate | 1 mg |
| | Total | 288 mg |

Process:

1. Rapid Expansion Supercritical Solution Technique was used for production of deferasirox Nanoparticles.
2. The solvent (carbon dioxide) was passed through a filter to a cooling system and allowed to liquefy and compressed with the desired pressure using an appropriate pump.
3. The liquid obtained in step (2) was allowed to enter the solution cell which contains deferasirox powder which was then sprayed to the nozzle.
4. The deferasirox powder obtained in step (3) was then blended with silicified MCC, sodium chloride and crospovidone.
5. The blend obtained in step (4) was lubricated using magnesium stearate and then compressed into tablets.

b) Tablets

| Sr. No | Ingredient | Qty |
|---|---|---|
| 1. | Deferasirox | 250 mg |
| 2. | Carbon Dioxide | q.s. |
| 3. | Acetone | q.s. |
| 4. | Silicified MCC | 190 mg |
| 5. | Sodium Chloride | 60 mg |
| 6. | Crospovidone | 74 mg |
| 7. | Magnesium Stearate | 2 mg |
| | Total | 576 mg |

Process:

1. Rapid Expansion Supercritical Solution Technique was used for production of deferasirox nanoparticles.
2. The solvent (carbon dioxide) was passed with acetone through a filter to a cooling system and allowed to liquefy and compressed with the desired pressure using an appropriate pump.
3. The liquid obtained in step (2) was allowed to enter the solution cell which contains deferasirox powder which was then sprayed to the nozzle.
4. The deferasirox powder obtained in step (3) was then blended with silicified MCC, sodium chloride and crospovidone.
5. The blend obtained in step (4) was lubricated using magnesium stearate and then compressed into tablets.

### Example 4

### Low Dose Nanoparticulate Deferasirox Using Solid Dispersion Technique—

a) Hard Gelatin Capsules

| Sr. No | Ingredient | Qty |
|---|---|---|
| 1. | Deferasirox | 125 mg |
| 2. | HPMC/HPC | 500 mg |
| 3. | Polyethylene Glycol | 50 mg |
| 4. | Purified Water | q.s. |
| 5. | Silicified MCC | 50 mg |
| 6. | Magnesium Stearate | 1 mg |
| | Total | 726 mg |

Procedure:

1. HPMC and polyethylene glycol were dissolved in water to obtain a clear solution
2. Deferasirox was then added to the solution obtained in step (2).
3. The suspension obtained in step (2) was spray drying to form a powder.
4. The deferasirox powder obtained in step (3) was then blended with silicified MCC (Prosolv SMCC 90).
5. The blend obtained in step (4) was then lubricated with magnesium stearate and filled into hard gelatin capsules.

b) Hard Gelatin Capsules

| Sr. No | Ingredient | Qty |
|---|---|---|
| 1. | Deferasirox | 125 mg |
| 2. | PVP K 30 | 500 mg |
| 3. | Polyethylene Glycol | 50 mg |
| 4. | Ethanol | q.s. |
| 5. | Silicified MCC | 50 mg |
| 6. | Magnesium Stearate | 1 mg |
| | Total | 726 mg |

Procedure:

1. PVP K 30 and polyethylene glycol were dissolved in ethanol to obtain a clear solution
2. Deferasirox was then added to the solution obtained in step (1).
3. The suspension obtained in step (2) was spray drying to form a powder.
4. The deferasirox powder obtained in step (3) was then blended with silicified MCC (Prosolv SMCC 90).
5. The blend obtained in step (4) was then lubricated with magnesium stearate and filled into hard gelatin capsules.

### Example 5

### Low Dose Nanoparticulate Deferasirox Using Nano Milling Technique—

a) Dispersible Tablets

| Sr. No. | Ingredients | Qty mg/tablet | |
|---|---|---|---|
| A) | Nano Milling | | |
| 1. | Deferasirox | 250 | |
| 2. | Sodium lauryl sulphate | 13.80 | |
| 3. | Hydroxypropylmethylcellulose | 50 | |
| 4. | Docusate Sodium | 5 | |
| 5. | Lactose Monohydrate | 50 | |
| 6. | Purified water | q.s | |
| B) | Dry Mix | | |
| 7. | Lactose Monohydrate | 180.02 | |
| 8. | Crospovidone | 50 | |
| C) | Blending & Lubrication | | |
| 9. | Sodium Chloride | 60 | |
| 10. | Crospovidone | 25 | |
| 11. | Microcrystalline cellulose, silicified | 194.18 | |
| 12. | Magnesium Stearate | 2 | |
| | Total | 880 | mg |

Process:

1. Docusate sodium, HPMC, sodium lauryl sulphate and lactose were solubilized in water
2. Deferasirox was dispersed in the solution obtained in step (1);
3. The dispersion obtained in step (2) was homogenized and then nanomilled.
4. Nanomilled drug slurry obtained in step (3) was adsorbed by spraying on lactose monohydrate and crospovidone mixture to form granules;
5. Granules obtained in step (4) were blended with sodium chloride, crospovidone and silicified microcrystalline cellulose and lubricated with magnesium stearate
6. Lubricated granules obtained in step (5) were compressed into tablets.

b) Dispersible Tablets

| Sr. No. | Ingredients | Qty mg/tablet | |
|---|---|---|---|
| A) | Nano Milling | | |
| 1. | Deferasirox | 300.00 | |
| 2. | Sodium lauryl sulphate | 16.56 | |
| 3. | Hydroxypropylmethylcellulose | 60.00 | |
| 4. | Docusate Sodium | 6.00 | |
| 5. | Lactose Monohydrate | 60.00 | |
| 6. | Purified water | q.s | |
| B) | Dry Mix | | |
| 7. | Lactose Monohydrate | 216.02 | |
| 8. | Crospovidone | 60.00 | |
| C) | Blending & Lubrication | | |
| 9. | Sodium Chloride | 72.00 | |
| 10. | Crospovidone | 30.00 | |
| 11. | Microcrystalline cellulose, silicified | 233.02 | |
| 12. | Magnesium Stearate | 2.40 | |
| | Total | 1056 | mg |

Process:

1. Docusate sodium, HPMC, sodium lauryl sulphate and lactose were solubilized in water
2. Deferasirox was dispersed in the solution obtained in step (1);
3. The dispersion obtained in step (2) was homogenized and then nanomilled.
4. Nanomilled drug slurry obtained in step (3) was adsorbed by spraying on lactose monohydrate and crospovidone mixture to form granules;
5. Granules obtained in step (4) were blended with sodium chloride, crospovidone and silicified microcrystalline cellulose and lubricated with magnesium stearate
6. Lubricated granules obtained in step (5) were compressed into tablets.

### Example 6

### Low Dose Nanoparticulate Deferasirox Using Hot Melt Extrusion—

| Sr. No. | Ingredients | Qty mg/tablet | |
|---|---|---|---|
| A) | Nano Milling | | |
| 1. | Deferasirox | 300.00 | |
| 2. | Sodium lauryl sulphate | 16.56 | |
| 3. | Lactose Monohydrate | 100.00 | |
| 4. | Copovidone | 100.00 | |
| B) | Blending & Lubrication | | |
| 5. | Sodium Chloride | 72.00 | |
| 6. | Crospovidone | 30.00 | |
| 7. | Microcrystalline cellulose, silicified | 233.04 | |
| 8. | Magnesium Stearate | 2.40 | |
| | Total | 854 | mg |

Process:

1. Deferasirox with mixed with sodium lauryl sulphate, lactose monohydrate and copovidone.
2. The blend obtained in step (1) was hot melt extruded.
3. The extrudes obtained in step (2) were sized to form granules.
4. The sized granules obtained in step (3) were blended with sodium chloride, crospovidone and silicified microcrystalline cellulose and lubricated with magnesium stearate
5. Lubricated granules obtained in step (4) were compressed into tablets.

### Example 7

### Pilot Study I

An open-label, balanced, randomized, two-treatment, two-sequence, two-period, single dose, crossover, comparative bioavailability study in healthy, adult, male human subjects under fasting conditions was performed.

Test Product (T): deferasirox 250 mg dispersible tablets, corresponding to example 5(a) (Particle size, D₉₀—0.282 μm) manufactured by Cipla Limited, India. Reference Product (R): EXJADE® 250 mg dispersible tablets, marketed by Novartis Europharm Limited, UK.

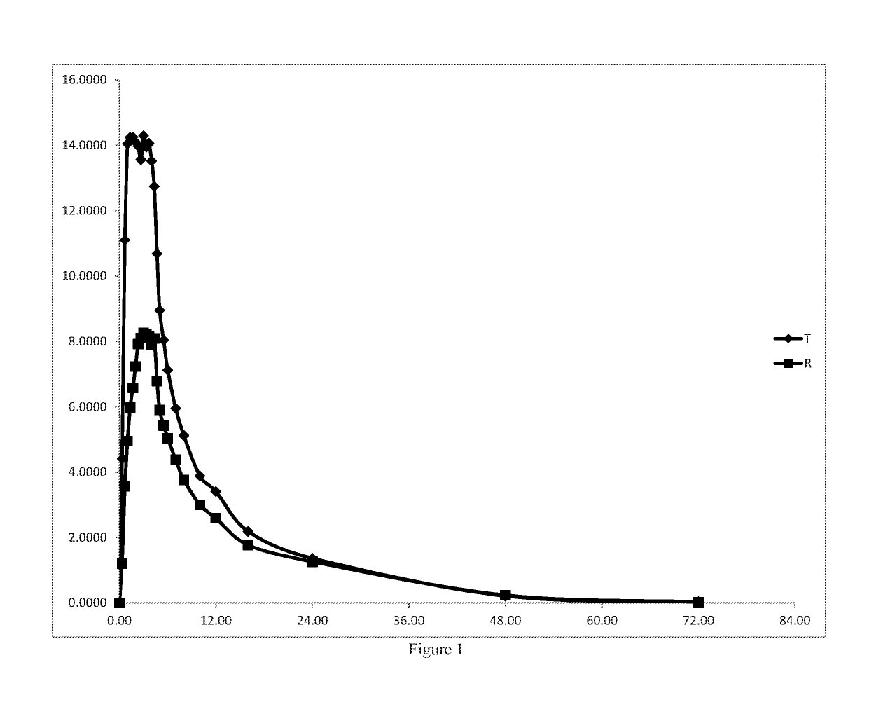

When the Test Product (T) was compared with the Reference Product (R) the Cₘₐₓ of the Test Product (T) was approximately 200% of the Cₘₐₓ of the Reference Product (R) and the AUC of the Test Product (T) was approximately 145% of the AUC of the Reference Product (R)—see FIG. 1.

### Example 8

### Pilot Study II

An open-label, randomized, three-treatment, three-sequence, three-period, single-dose, crossover, comparative bioavailability study in healthy, non-smoking male and female human subjects under fasting conditions was performed.

Test Product (T1) [Test A]: deferasirox 175 mg dispersible tablets (Particle size, D₉₀—0.298 μm), and (T2) [Test B]: deferasirox 250 mg dispersible tablets, corresponding to example 5(a) (Particle size D₉₀—0.298 μm) manufactured by Cipla Limited, India were compared with the Reference Product (R): EXJADE® 500 mg dispersible tablets, marketed by Novartis Europharm Limited, UK.

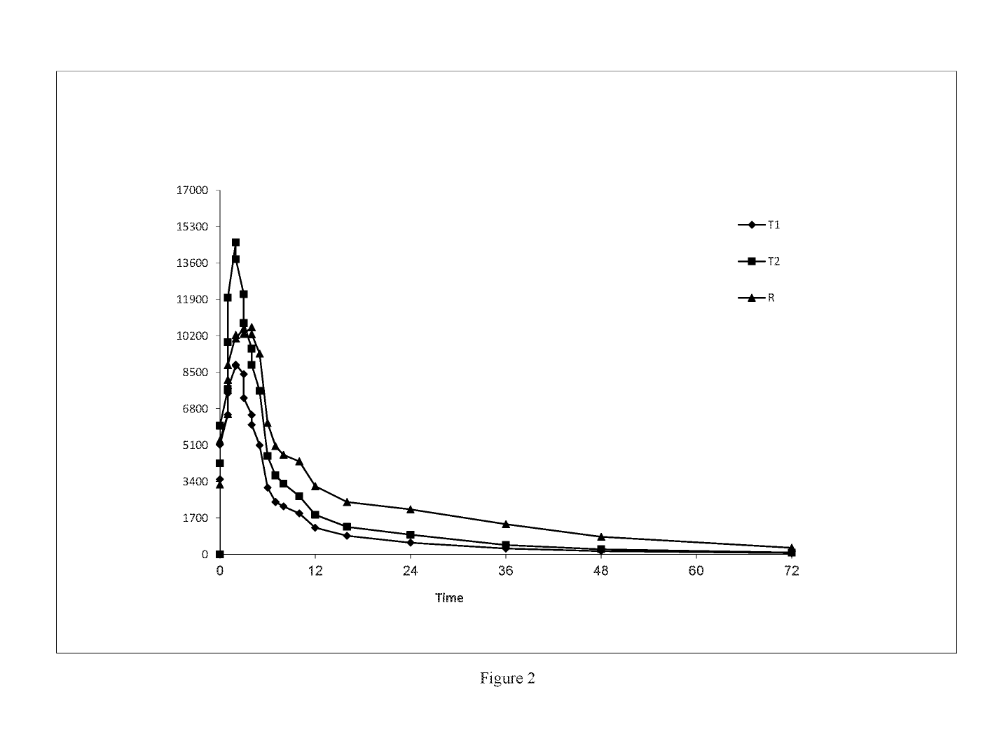

When (T1) and (T2) were compared with the Reference Product (R) the results indicated a significantly lower AUC (33%) than the AUC of the Reference Product (R), however the Cₘₐₓ was high (˜27%) for (T2) while (T1) exhibited a considerably lower AUC than the Cₘₐₓ, of the Reference Product—see FIG. 2.

### Example 9

### Pilot Study III

An open-label, balanced, randomized, four-treatment, four-sequence, four-period, single-dose, crossover comparative oral bioavailability study in normal, healthy, adult, human subjects under fasting condition was performed.

Test Products: (T1) deferasirox 300 mg dispersible tablets, corresponding to example 5(b) (Particle size D₉₀ —2.63 μm), (T2): deferasirox 250 mg dispersible tablets, corresponding to example 5(a) (Particle size D₉₀ —2.63 μm), and (T3): deferasirox dispersible tablets 375 mg (Particle size D₉₀ —0.3 μm and D₉₀ —28 μm) manufactured by Cipla Limited, India were compared with the Reference Product (R): EXJADE® 500 mg dispersible tablets marketed by Novartis Europharm Limited, UK.

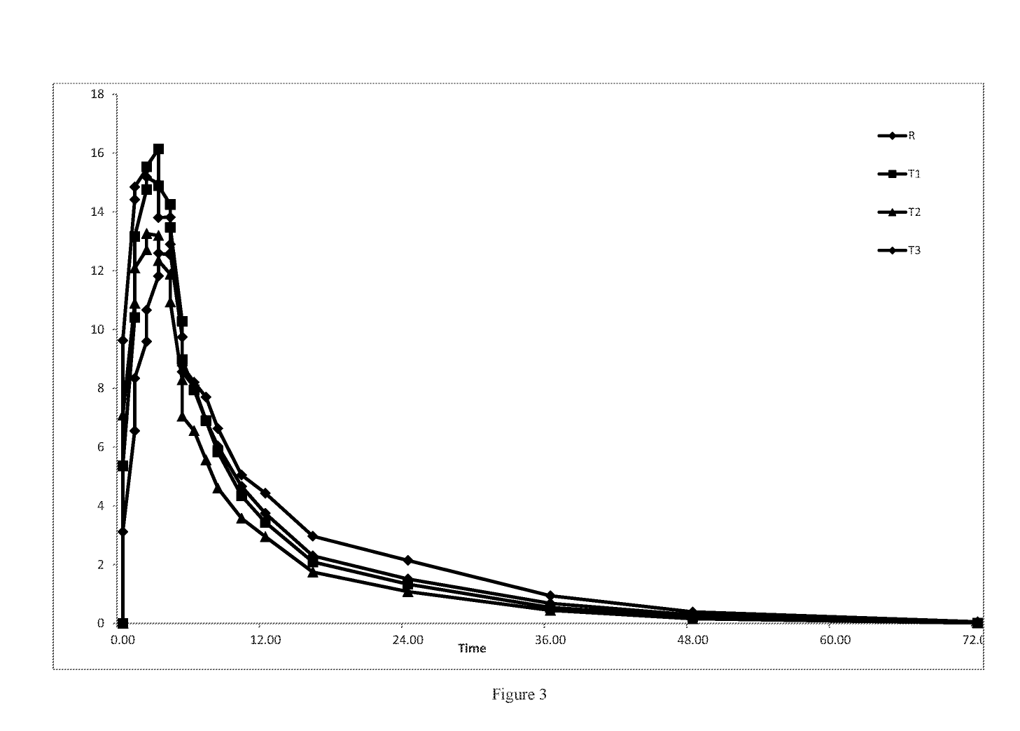

This study indicated that (T2) exhibited satisfactory Cₘₐₓ results but exhibited slightly lower levels for AUC in comparison with the AUC of the Reference Product, whereas (T1) and (T3) exhibited considerably higher Cₘₐₓ than the Cₘₐₓ of the Reference Product (R)—see FIG. 3.

### Example 10

### Pilot Study IV

An open-label, randomized, two-treatment, four-sequence, four-period, single-dose, crossover, comparative bioavailability study in healthy, adult, human subjects under fasting and fed conditions was performed.

Test Product (T): deferasirox 250 mg dispersible tablets, corresponding to example 5(a) (Particle Size D₉₀—2.63 μm) manufactured by Cipla Limited, India was compared with the Reference Product (R): EXJADE® 500 mg Dispersible Tablets marketed by Novartis Europharma UK.

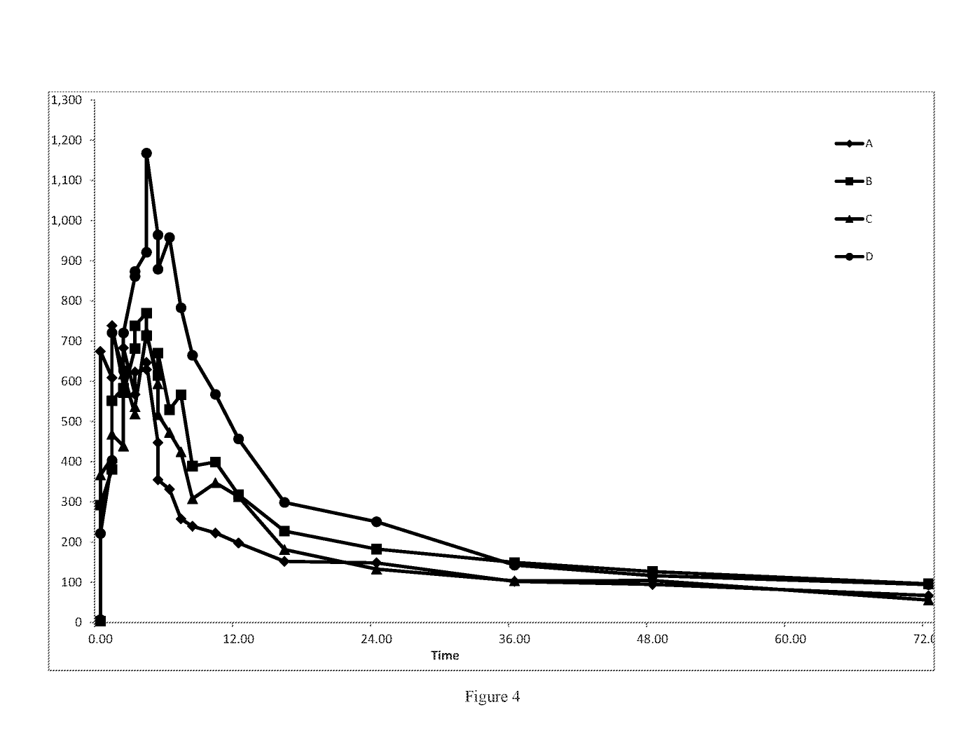

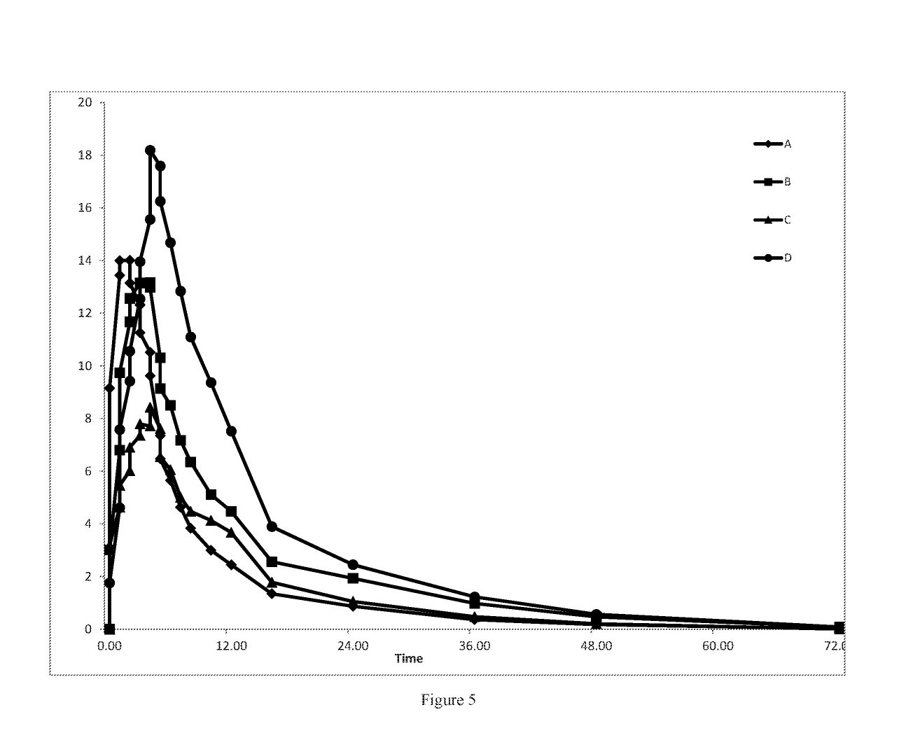

The food effect study indicates that there is no significant increase in Cmax for the Test Product (T) when compared to that of the Reference Product (R)—see FIGS. 4 and 5 in which “A” represents the Test Product (T) under fasting condition, “B” represents the Reference Product (R) under fasting condition, “C” represents the Test Product (T) under fed condition, and “D” represents the Reference Product under fed condition.

It will be readily apparent to one skilled in the art that varying substitutions and modifications may be made to the invention disclosed herein. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “an excipient” includes a single excipient as well as two or more different excipients, and the like.

What is claimed is:

1. A method for the treatment of chronic iron overload in a human patient wherein the method comprises administering an effective amount of a pharmaceutical composition comprising deferasirox or a pharmaceutically acceptable derivative thereof in a daily dose of from about 1 mg/kg to less than about 20 mg/kg of body weight.

2. The method of claim 1, wherein the chronic iron overload includes blood transfusion dependent thalassemia or non-transfusion dependent thalassemia (NTDT).

3. The method of claim 2, wherein the method comprises administering an effective amount of a pharmaceutical composition comprising deferasirox or a pharmaceutically acceptable derivative thereof in a daily dose of from about 2 mg/kg to less than about 20 mg/kg of body weight.

4. The method of claim 3, wherein the method comprises administering an effective amount of a pharmaceutical composition comprising deferasirox or a pharmaceutically acceptable derivative thereof in a daily dose of from about 3 mg/kg to less than about 15 mg/kg of body weight.

5. The method of claim 1, wherein the pharmaceutically acceptable derivative of deferasirox is a salt, solvate, complex, hydrate, isomer, ester, tautomer, anhydrate, enantiomer, polymorph or prodrug.

6. The method of claim 1, wherein said pharmaceutical composition is for oral administration.

7. The method of claim 6, wherein said pharmaceutical composition is in the form of a tablet.

8. The method of claim 7, wherein said tablet is in the form of a dispersible tablet or a film-coated tablet.

9. The method of claim 7, wherein said tablet comprises from about 50 mg to about 100 mg of deferasirox.

10. The method of claim 9, wherein said tablet comprises from about 150 mg to about 200 mg of deferasirox.

11. The method of claim 10, wherein said tablet comprises from about 260 mg to about 350 mg of deferasirox.

12. The method of claim 6, wherein the deferasirox or a pharmaceutically acceptable derivative thereof is in the form of particles having an average particle size of greater than about 1 μm but less than or equal to about 30 μm.

13. The method of claim 12, wherein the deferasirox or a pharmaceutically acceptable derivative thereof is in the form of particles having an average particle size of greater than about 1 μm but less than or equal to about 8 μm.

14. The method of claim 7, wherein the tablet comprises a surfactant, viscosity enhancing agent, solubilizer, an anticaking agent, a buffer, a polymer, a sweetener, solvents, co-solvents, a vehicle, a carrier, an adsorbent, a channeling agent, an opacifier, a diluent, a filler, a glidant, an anti-adherent, a binder, a disintegrant and a lubricant.

15. The method of claim 14, wherein the surfactant is an amphoteric, non-ionic, cationic or anionic surfactant or combinations thereof.

16. A pharmaceutical composition comprising deferasirox or a pharmaceutically acceptable derivative thereof for use in the treatment of chronic iron overload, wherein said composition comprises the deferasirox or a pharmaceutically acceptable derivative thereof in an amount effective to provide a daily dose of from about 1 mg/kg to less than about 20 mg/kg of body weight when administered to a patient requiring treatment for chronic iron overload.
